(19) United States
(12) Patent Application Publication
(10) Pub. No.: US 2019/0000339 A1
(43) Pub. Date: Jan. 3, 2019

(54) SYSTEMS, APPARATUS, AND METHODS FOR ELECTRO-ANATOMICAL MAPPING OF A CATHETER WITH ELECTRODE CONTACT ASSESSMENT AND ROTOR PROJECTION

(71) Applicant: CardioNXT, Inc., Westminster, CO (US)

(72) Inventors: Paul KESSMAN, Lakewood, CO (US); Jerome EDWARDS, Erie, CO (US); Bao NGUYEN, Westminster, CO (US)

(21) Appl. No.: 15/880,966

(22) Filed: Jan. 26, 2018

Related U.S. Application Data
(63) Continuation of application No. PCT/US2016/044228, filed on Jul. 27, 2016.
(60) Provisional application No. 62/197,263, filed on Jul. 27, 2015.
(60) Provisional application No. 62/197,267, filed on Jul. 27, 2015.
(60) Provisional application No. 62/197,276, filed on Jul. 27, 2015.

(51) Int. Cl. A61B 5/0408; A61B 5/00; A61B 5/044

(52) U.S. Cl. CPC A61B 5/04085; A61B 5/4836; A61B 5/046; A61B 5/6823; A61B 5/044

(57) ABSTRACT

A system includes a pair of external body electrodes, a first control unit and a second control unit. The first control unit is arranged to provide a constant current at a first frequency across the pair of external body electrodes coupled to a body of a patient. The first control unit further arranged to provide a constant voltage circuit across the body of the patient at a second frequency different from the first frequency. The second control unit is arranged to measure a voltage of an internal electrode located within a chamber of a heart of the patient in the first frequency. The second control unit further arranged to measure a voltage of the internal electrode in the second frequency to determine a voltage change.

### CROSS-REFERENCE TO RELATED APPLICATIONS

This application is a continuation of International Patent Application No. PCT/US2016/044228, entitled “Systems, Apparatus, and Methods for Electro-Anatomical Mapping of a Catheter With Electrode Contact Assessment and Rotor Projection,” filed on Jul. 27, 2016, which claims priority to and the benefit of U.S. Provisional Patent Application No. 62/197,263, entitled “Deformable Heart Template Registration,” filed on Jul. 27, 2015; U.S. Provisional Patent Application No. 62/197,267, entitled “Methods, Apparatuses, and Systems for Measuring Rotation of Catheter with an Electropotential Localizer,” filed on Jul. 27, 2015; and U.S. Provisional Patent Application No. 62/197,276, entitled “Methods, Apparatuses, and Systems for Electro-Anatomical Mapping of Basket Catheter with Electrode Contact Assessment and Rotor Projection,” filed on Jul. 27, 2015; each of which is incorporated herein by reference in its entirety.

### BACKGROUND

The embodiments described herein generally relate to aiding physicians in performing surgical procedures on patients and more particularly to systems, apparatus and methods for non-invasively mapping the electrical activity of the heart and identifying sources of arrhythmia using electrodes on the patient's external body surface, projecting that information onto a computer imaged three-dimensional or four-dimensional model of the heart, and co-registering the model based on cardiac activity information with a position localization system which can be used to accurately navigate instruments during a procedure with respect to the sources of arrhythmia for treatment of the patient, and further augmenting the cardiac activity information with real-time intracardiac recordings from instruments navigated during the surgical procedure.

There exist very complex cardiac arrhythmias such as Atrial Fibrillation that are extremely hard to deconstruct to a source with conventional intracardiac catheters and traditional twelve-lead electrocardiogram readings. There are high resolution cardiac electrogram processing techniques that utilize large numbers of sampling electrodes spread all over a patient's thorax along with imaging techniques such as computerized tomography or magnetic resonance imaging to create models of the heart and project electrical activity at the body surface onto these imaging models. Ultimately, to make use of this high resolution body surface cardiac electrogram information for treating patients, the information must be presented in a manner in which the surgeon can process that information during a surgery, identify sources of the arrhythmia, translate that arrhythmia source information to anatomic information that they are able to manipulate and then deliver therapy to that source to treat the patient. Further, the electrical activity of the heart is constantly changing and measurements acquired from outside the body must be augmented with measurements acquired from inside the body to best highlight potential anatomic source regions of the arrhythmia. This must all be performed in a stable and consistent way, over heart beat and respiration cycles, so that the surgeon can trust the information before they deliver therapy to a particular region of the heart. Serious complications, such as sudden cardiac arrest, stroke, atrio-esophageal fistula and perforation can occur if therapy is delivered internally inaccurate based on the body surface cardiac electrical information.

Therefore, a practical need exists to have an accurate way of delivering body surface cardiac electrical information to a surgeon during a procedure in which they are manipulating instruments inside the body to diagnose the source of an arrhythmia and deliver therapy to that source.

### SUMMARY OF THE INVENTION

In an embodiment, a system includes a pair of external body electrodes, a first control unit and a second control unit. The first control unit is arranged to provide a constant current at a first frequency across the pair of external body electrodes coupled to a body of a patient. The first control unit further arranged to provide a constant voltage circuit across the body of the patient at a second frequency different from the first frequency. The second control unit is arranged to measure a voltage of an internal electrode located within a chamber of a heart of the patient in the first frequency. The second control unit further arranged to measure a voltage of the internal electrode in the second frequency to determine a voltage change.

### BRIEF DESCRIPTION OF THE DRAWINGS

FIG. 1 illustrates an electrical cardiac activity mapping system, according to an embodiment.

FIG. 2 illustrates the electrical cardiac activity mapping system of FIG. 1 applied to a patient.

FIGS. 3-7 illustrate circuit diagrams of the electrical cardiac activity mapping system of FIG. 1.

FIG. 8 illustrates an actual basket catheter and a computer generated rendition of the basket catheter, according to an embodiment.

FIG. 9 illustrates a portion of the computer generated rendition of the basket catheter of FIG. 8 in contact with a heart chamber.

FIG. 10 illustrates an electroanatomical rotor map projected on 3-dimensional geometry, according to an embodiment.

FIG. 11 illustrates in side view a distal portion of a catheter having separate radial electrodes, and FIG. 12 shows a cross-sectional view of the distal portion of the catheter of FIG. 11, according to an embodiment.

FIG. 13 illustrates a deformable template, according to an embodiment.

FIG. 14 illustrates an attractive force exerted by a centroid of the model template of FIG. 13, and an equation for determining the attractive force of the centroid of the template.

FIG. 15 illustrates a repulsive force of the point cloud to the surface of the model template of FIG. 13, and shows equations for determining the repulsive force.

FIG. 16 illustrates a repulsive force of sub-model segments of the template model of FIG. 13 pushing against each other, and shows equations for determining the repulsive force of the sub-model segments.

FIG. 17 illustrates the template of FIG. 13 in a final configuration.

FIG. 18 shows a flow diagram of a method of electro-anatomical mapping, according to an embodiment.

### DETAILED DESCRIPTION

In some embodiments, physicians can utilize multiple independent data components, each data component providing diagnostic clinical data about a patient. For example, a first data component can include an electrocardiogram (EKG) map that can identify an atrial tachycardia. A second data component can include an instrument position map that identifies the positions of instruments inside the heart based on a position sensing system that can aid the physician in manipulating instruments during an electrophysiology study. The electrocardiogram map can help localize the region within the atria identified as the source of ectopic activity driving a cardiac arrhythmia. Catheters can be navigated, using the instrument position map, to that region of the heart. Further information can be gathered from that region of the atrium, such as, for example, local activation and voltage, to identify the specific ectopic pathologic source to treat.

In some embodiments, a system includes one or more pairs of external body electrodes that can be coupled to a patient's body, a first control unit and a second control unit. The first control unit can provide constant current at a first frequency across the one or more pairs of external body electrodes when the external body electrodes are coupled to the patient's body. The first control unit can further provide a constant voltage circuit across the patient's body at a second frequency different from the first frequency. The second control unit can measure a voltage in the first frequency of an internal electrode located within a chamber of a heart of the patient. The second control unit can further measure a voltage of the internal electrode in the second frequency to determine a voltage change.

In some embodiments, a system includes a trackable medical instrument and a control unit operably coupled to the trackable medical instrument. The control unit can receive positional data from the trackable medical instrument when the trackable medical instrument is disposed within a patient's heart chamber. The control unit can further generate a cloud of points in at least three dimensions based on locations visited by the trackable medical instrument within the heart chamber. The control unit can further modify a template three-dimensional surface model of a generic heart chamber based on interactive forces between the cloud of points and the template three-dimensional surface model.

In some embodiments, a method includes collecting location points within a patient's heart anatomy. The method further includes calculating an attractive force of points in the plurality of location points to a centroid of a template and calculating a repulsive force of the plurality of location points to the template. The template includes multiple template regions. The method further includes recursively balancing the attractive force and the repulsive force to equilibrium, and overlaying a modified template over the location points. The method further includes segmenting a plurality of point clouds based on the modified template.

In some embodiments, an apparatus includes an elongated cylindrical catheter and a control unit. The catheter includes an array of electrodes aligned radially around the outer circumference of the catheter. At least two electrodes from the array of electrodes are partially wrapped around opposite ends of the circumference of the catheter, and each electrode from the array of electrodes is independently connected to a navigation system. The control unit can receive positional data of each electrode from the array of electrodes. The control unit can further define a vector orthogonal to a center axis of the catheter, and calculate a roll of the catheter orientation.

In some embodiments, a system is used for receiving diagnostic information and/or for delivering therapy to a body, e.g., a chamber of a human heart. For example, with a medical instrument inserted into and disposed within a chamber of the heart, the system is used for detecting contact of the medical instrument with cardiac tissue, sending electrical signals to the heart for diagnostic purposes (e.g., pacing), and/or providing therapy to cardiac tissue (e.g., ablating defective cardiac tissue).

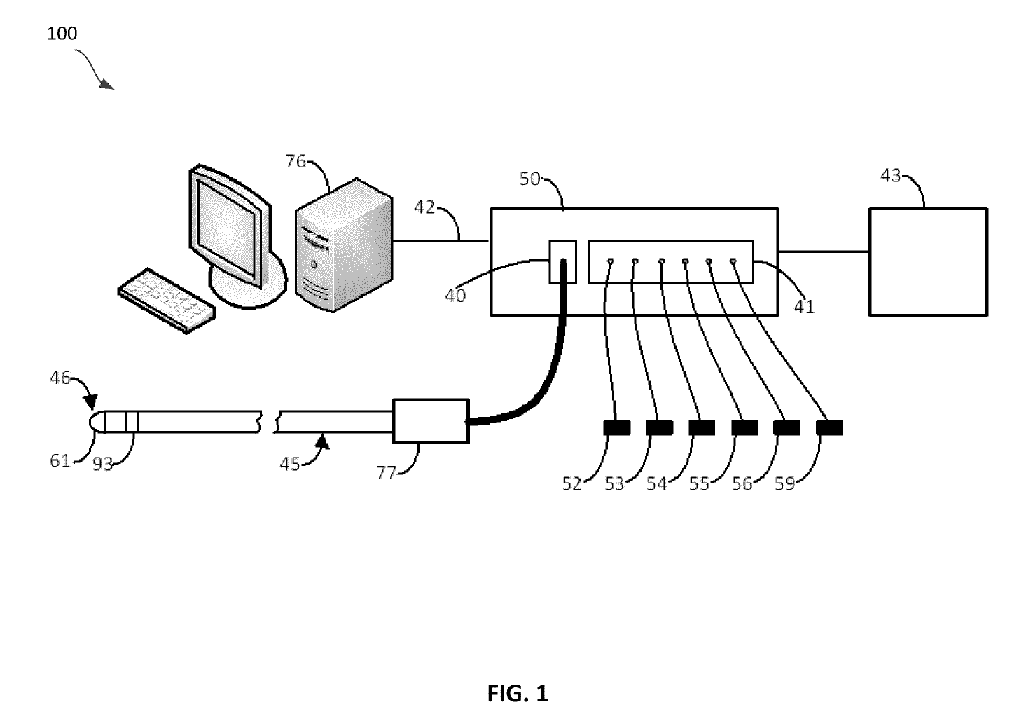

For example, FIG. 1 is a schematic illustration of such a system, according to an embodiment. As shown, system 100 includes a catheter 45 configured for insertion into a body (e.g., into a chamber of a human heart). The catheter 45 includes a contact electrode 61 located at a distal end or tip of the catheter 45 for measuring electrical information, properties, characteristics, or the like of body tissue, e.g., electrical properties of heart tissue. Additionally, or alternatively, the contact electrode 61, in some instances, is used for sending and/or delivering electrical signals to body tissue for diagnostic purposes (e.g., pacing), and/or for therapeutic purposes (e.g., ablating defective cardiac tissue). In yet further instances, the contact electrode 61 can be used to detect contact of the contact electrode 61 with a target tissue (e.g., cardiac tissue). Although the contact electrode 61 is shown and described in this embodiment as being located at the distal tip of the catheter 45, in alternative embodiments, the contract electrode can be located at any suitable portion of the catheter (e.g., proximal to the distal end of the catheter).

In some embodiments, a catheter can optionally include one or more proximal electrodes any suitable purpose. For example, in this embodiment, the catheter 45 includes a proximal electrode 93 located proximal to the contact electrode 61. The proximal electrode 93 can be used for diagnostic purposes, e.g., electrogram recording, and/or measuring and/or detecting contact with target body tissue. In some instances, for example, both the contact electrode 61 and the proximal electrode 93 can be used in conjunction with each other to measure and/or detect contact with target body tissue.

A proximal end portion of the catheter 45 includes a handle 77 that can be used by the user/operator to manipulate movement of the catheter 45. For example, in some instances, the handle 77 can include mechanisms that can be manipulated by an operator to steer the catheter 45 in a desired direction and/or to position and/or orient the catheter 45 as desired.

As shown in FIG. 1, the catheter 45 is communicatively coupled to a signal processing circuit 50 (also referred to herein as “signal processor”). The signal processor 50 is configured to receive, amplify, filter, and/or digitize signals generated by and/or received from the catheter 45 (e.g., signals generated from contact electrode 61 and/or proximal electrode 93). The signal processor 50 is further configured to compute or otherwise determine a position and/or orientation of the catheter 45, and/or electrical information, properties, and/or characteristics of, for example, a heart chamber, based on information collected and provided by the catheter 45.

The system 100 further includes a console 76 having a computer and an image display device. The console 76 provides controls to operate the system 100, e.g., to start and stop collection of data from the catheter 45. The console 76 can use the electrical and/or location information received by the catheter 45 (e.g., the contact electrode 61 and/or the proximal electrode 93) and/or the signal processor 50 to generate, render, and/or otherwise display a representation of such information. For example, in some instances, the console 76 can generate and display a graphical representation of the information, such as, for example, an electrical or electroanatomical map of a chamber of the heart of the patient 57.

As shown in FIG. 1, the system further includes six patient electrode contacts 52, 53, 54, 55, 56, 59. Each patient electrode contact is communicatively coupled to the signal processor 50, and is configured to transmit signals into and/or through the human body to localize the contact electrode 61, the proximal electrode 93 and/or other catheters and/or electrode used in the procedure and disposed within the patient.

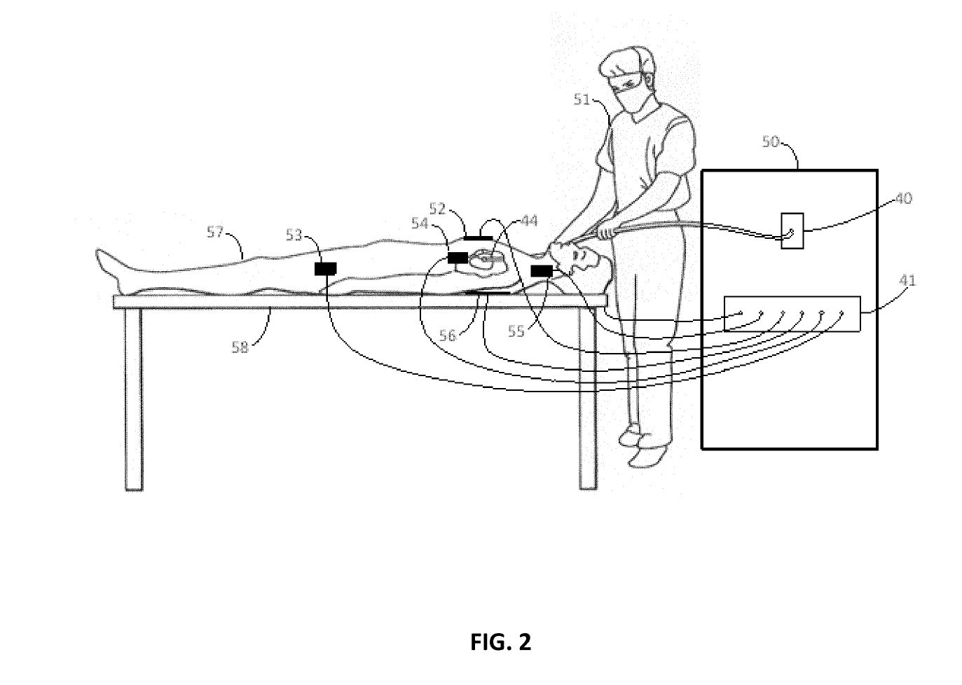

With six patient electrode contacts selectively located on the patient, three orthogonal axes can be generated. FIG. 2 illustrates such placement of the six patient electrode contacts on a patient 57. As shown, the patient electrode contacts 52, 53, 54, 55, 56 and 59 (not shown in FIG. 2) are placed at the following anatomical locations, respectively: chest, leg, left side, neck, back, right side. The three orthogonal axes are formed by transmitting three separate frequency currents into the patient 57 through pairs of electrode. For example, signals are transmitted through patient electrode contacts 54 (left side) and 59 (right side) at a same frequency (e.g., a carrier frequency) and at phases different from each other, thereby generating a resultant current that forms a voltage through the patient 57 which is then received by both the contact electrode 61 and the proximal electrode 93 to determine the location of the contact electrode 61 and the proximal electrode 93 within the patient's 57 heart. Signals are similarly transmitted through patient electrode contacts 52 (front) and 56 (back) at a same frequency (e.g., a frequency different from the carrier frequency) and at phases different from each other, and through patient electrode contacts 55 (neck) and 53 (leg) at a same frequency and at phases different from each other. Location coordinates associated with the patient electrode contacts can be processed at the signal processor 50 and then sent to the console 76 for representative display and/or generation and display of electroanatomic maps.

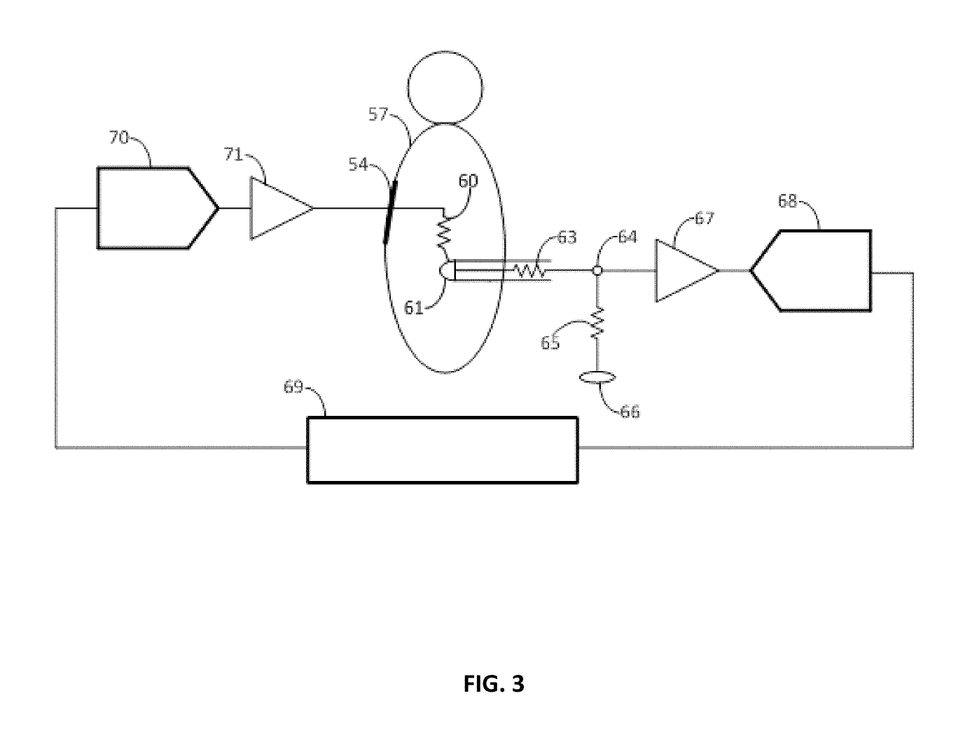

The system 100 further includes a signal processor controller 69 configured to control generation and/or transmission of the localization frequencies, amplitude, and phase. As shown in FIG. 3, the signal processor controller 69 is communicatively coupled to a digital-to-analog (DTA) converter 70, an amplifier 71, and to the patient electrode contact 54. For ease of illustration in FIG. 3, only one patient electrode contact is shown and described, however, it should be understood that the signal processor controller 69 can transmit signals to any number of patient electrode contacts (e.g., all of the patient electrode contacts coupled to a particular patient). For example, as shown in FIG. 5, a signal is sent by the signal processor controller 69 to the patient electrode contact 52 via the DTA converter 74 and amplifier 75, to the patient electrode contact 59 via the DTA converter 72 and amplifier 73, and to the patient electrode contact 54 via the DTA converter 70 and amplifier 71.

Referring for simplicity to FIG. 3, in use, the signal processor controller 69 can be used to transmit a digital signal to the DTA converter 70. The DTA converter 70 can convert the digital signal to an analog signal, and then send the analog signal through the amplifier 71 to amplify the analog signal such that the patient electrode contact 54 receives the amplified signal.

As shown in FIG. 3, the catheter 45 is communicatively coupled to an input amplifier 67, an analog-to-digital (ATD) converter, and the signal processor controller 69. In this manner, in use, the signal generated at and/or provided by the contact electrode 61 of the catheter 45 (e.g., disposed within the patient 57) is transmitted through the input amplifier 67 to amplify the signal. From the input amplifier 67, the amplified signal is transmitted through the ATD converter 68 to convert the analog signal to a digital signal. The digital signal is then transmitted from the ATD converter to the signal processor controller 69. Referring to FIG. 3, the voltage at the input amplifier terminal 64, measured with reference to the circuitry internal reference 66, is proportional to the body-to-contact impedance 60, electrode impedance 63, and input amplifier impedance 65.

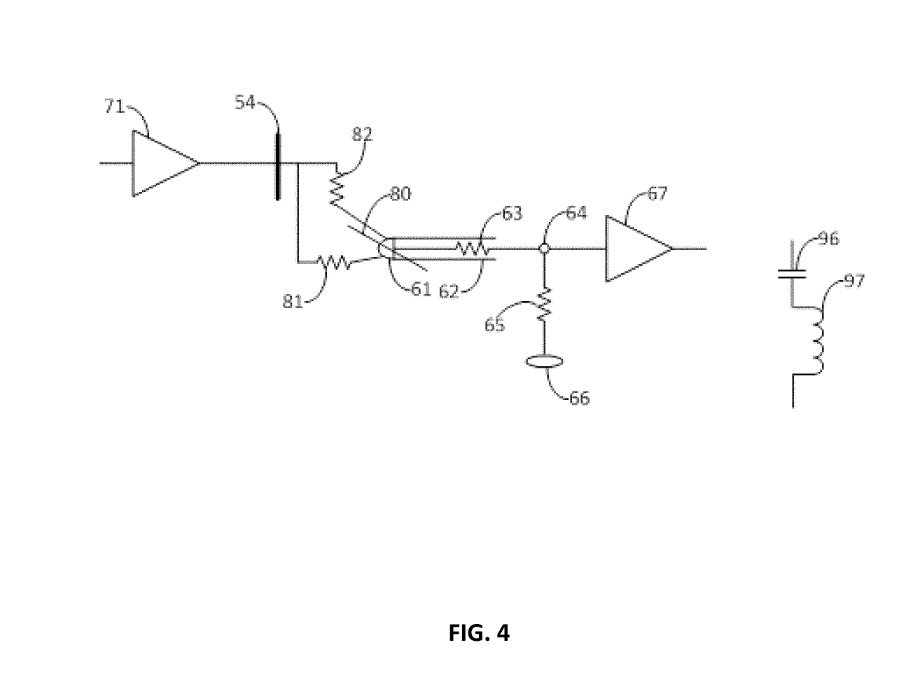

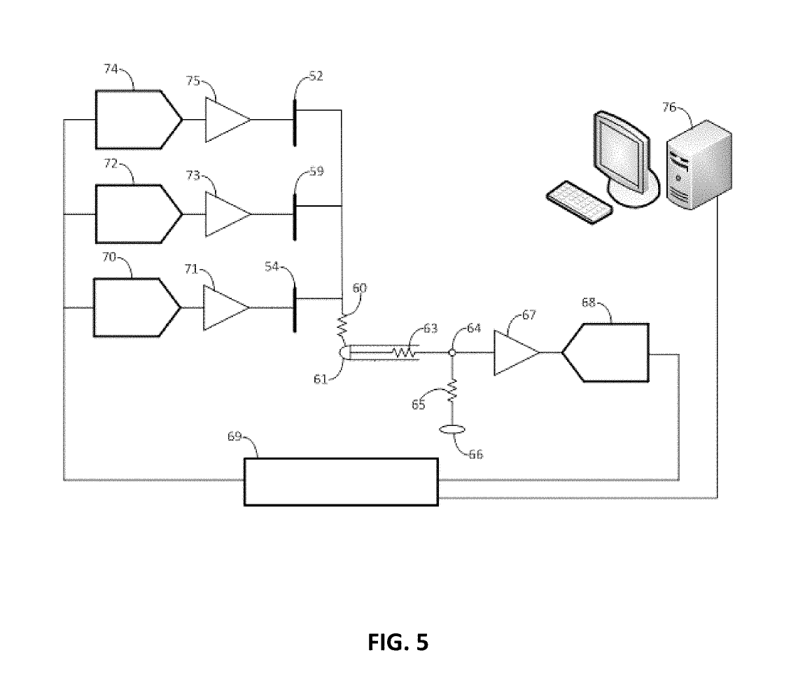

The contact impedance 60 can be computed based on the electrode impedance 63 and the input amplifier impedance 65. FIG. 4 illustrates the contact impedance measurement in greater detail. As shown in FIG. 4, the contact impedance 60 is represented by two constitute parts, i.e., a tissue-contact impedance 81 and a blood-contact impedance 82, and separated by a heart/tissue boundary 80. Resistance based on contact between the tissue and the contact electrode 61 can be computed by the signal processor 69 and then transmitted to the console 76 for graphical display (e.g., in relation to an electroanatomical map).

For example, the following equation can be used by the signal processor 69 to compute the tissue resistance:

$$tissue resistance = \frac{( Rblood * Rmeasured )}{Rblood - Rmeasured}$$

Rblood represents the resistance of the blood of the patient. In some instances, for example, Rblood can be derived from the contact electrode 61 by placing the contact electrode 61 in contact with blood (e.g., by placing the catheter 45 in a blood pool. As another example to determine Rblood, an operator can maneuver the catheter 45 (and in turn the contact electrode 61) within the patient to a suitable location (e.g., such that the contact electrode 61 is in contact with blood and not tissue) with assistance from an electroanatomical map (e.g., of the heart and/or the heart surface) provided by the console 76.

Rmeasured represents a measured resistance. The following equation, for example, can be used by the signal processor 69 to compute the measured resistance:

$$Rmeasured = \frac{Rin * Vdrive}{Vin} - Relectrode - Rin$$

Rin represents the input impedance 65 of the input amplifier 67, Vdrive represents the voltage transmitted into the body by the amplifier 71, Relectrode represents the electrode impedance 63, and Vin represents the voltage measured by the ATD converter 68 and computed by the signal processor controller 69.

In use, the input amplifier impedance 65 is a complex impedance that can vary with frequency variations. As such, the signal processor controller 69 can help to improve sensitivity of the tissue contact measurement by transmitting frequencies for which the complex input impedance 65 is reduced and/or lower. For example, frequencies in the range of about 10 khz to about 100 khz can be used to improve or otherwise facilitate accurate tissue contact measurements.

In alternative embodiments, a specific complex input impedance 65 can be selected. For example, as illustrated in FIG. 5, the system 100 can include a tuned circuit including a capacitor 96 and an inductor 97 tuned for a particular frequency transmitted from the signal processor controller 96.

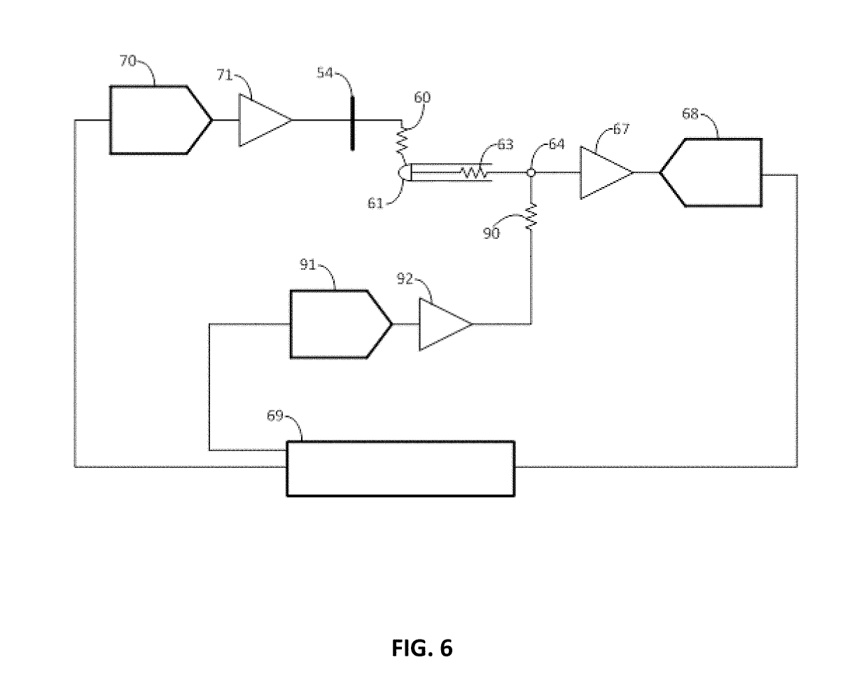

In further alternative embodiments, the amplifier impedance 65 can be modified through an active circuit including a DTA converter 91 and an amplifier 92. More specifically, as shown in FIG. 6, the signal processor controller 69 can modulate a frequency and/or phase of a signal across the circuit to alter the input impedance 90 such that the equivalent input impedance 65 is lower, thereby improving the sensitivity of the tissue contact measurements.

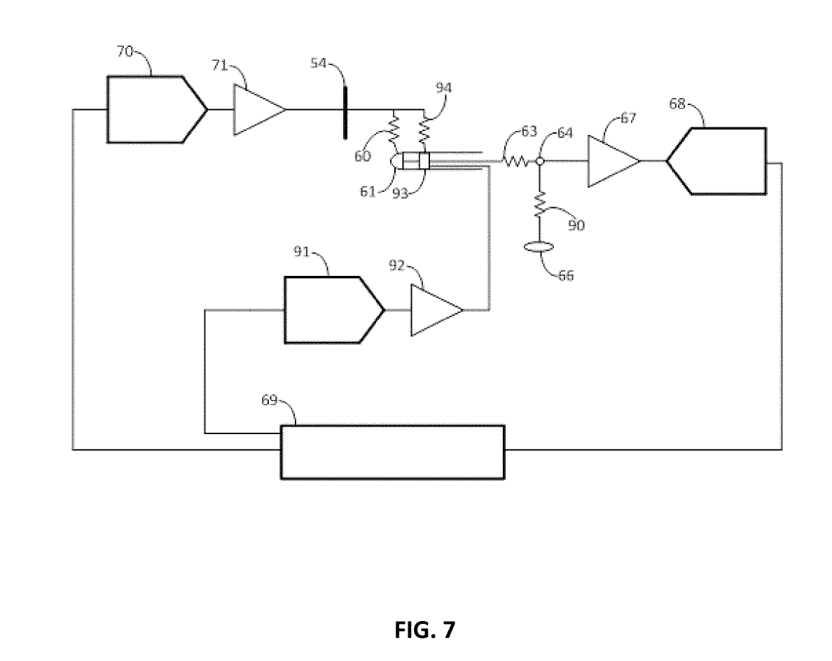

In yet further alternative embodiments, a contact measurement current can be transmitted from the proximal electrode 93, as shown for example in FIG. 7. In this manner, variation of blood impedance 82 can be measured (which may vary with the volume of blood surrounding the contact electrode 61 and the proximal electrode). In use, a signal can be generated and/or transmitted by the signal processor controller 69, converted to analog by the DTA converter 91, amplified at the amplifier 92, and then received by the proximal electrode 93.

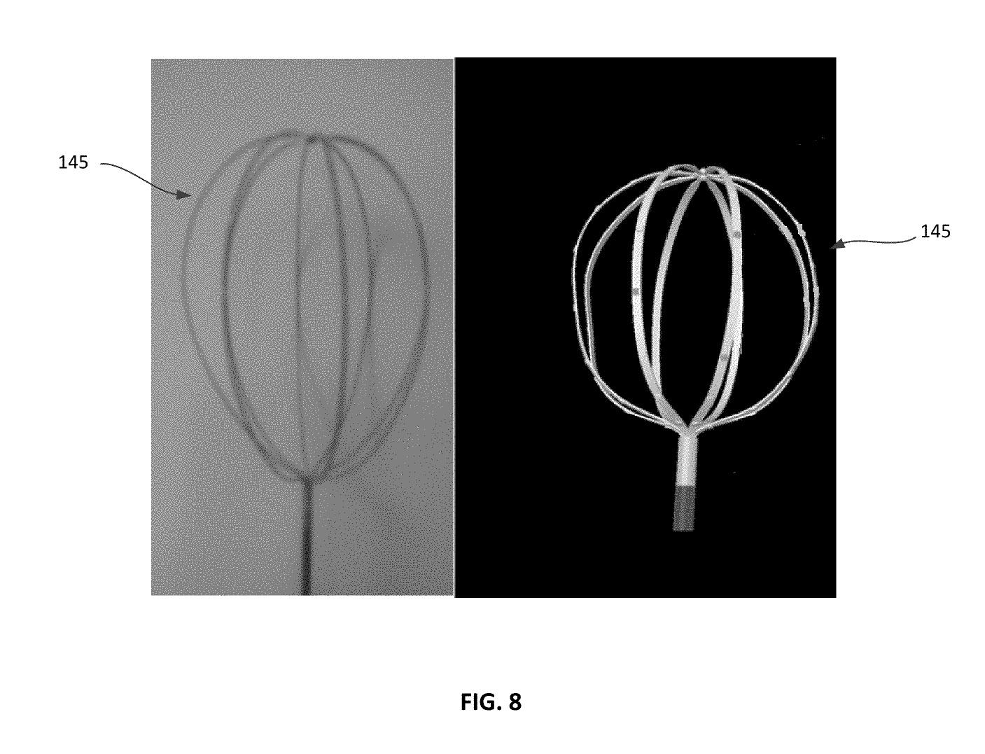

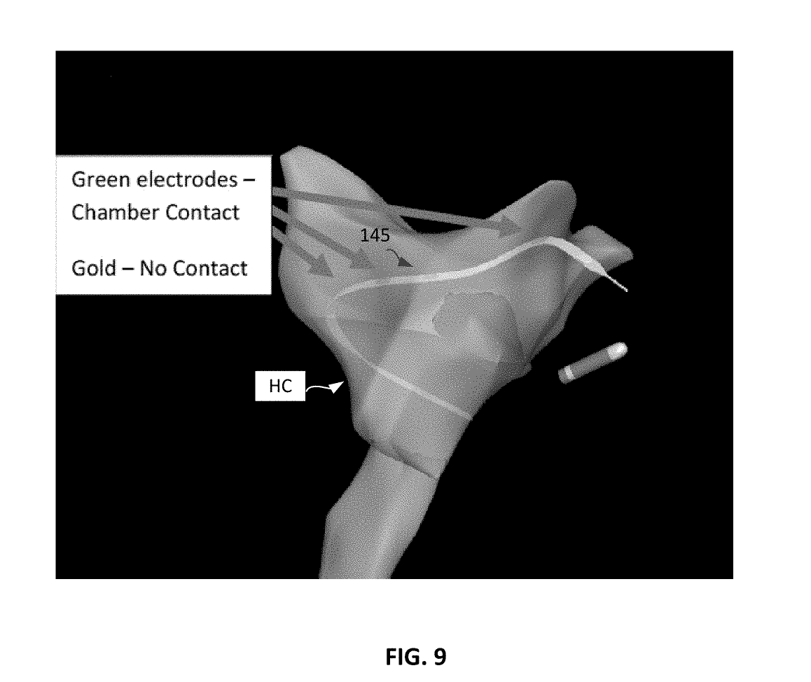

As described above with reference to FIG. 1, any suitable catheter can be used with the systems described herein. For example, as shown in FIG. 8, a basket catheter 145 can be used. On the left side of FIG. 8 is an image of an actual basket catheter, and on the right side of FIG. 8 is a computer generated rendition of the basket catheter 145 based on information obtained by the electropotential localization of the basket electrodes of the basket catheter 145 and known mechanical information of the catheter 145. Splines of the catheter 145 in the computer generated model can be deformed, representing the actual shape of the basket catheter 145 when it is inside of the patient. As illustrated in FIG. 9, a portion of the basket catheter 145 is in contact with a heart chamber HC. Specifically, the electrodes represented by green markings indicate actual contact of that portion of the basket catheter 145 being in contact with an adjacent portion of the heart chamber HC, and the electrodes represented by gold markings indicate no contact (e.g., based on a measurement having a value below a predefined contact threshold value) with an adjacent portion of the heart chamber HC.

In use, electrode contact is important during basket catheter 145 placement to ensure that a suitable number of electrodes are in contact with a target tissue, e.g., the heart chamber wall. Such contact can be determined by measuring the electrode impedance, as such impedance increases in response to contact with tissue. Any suitable threshold can be set (e.g., at the console 76) such that when the measured impedance meets and/or exceeds the threshold that graphical representation of the electrode associated with the impedance can change colors to indicate contact. Although shown and described as a change in color, any suitable distinctive graphical representation between electrodes that meet and/or exceed the threshold and electrodes that do not meet and/or exceed the threshold can be used.

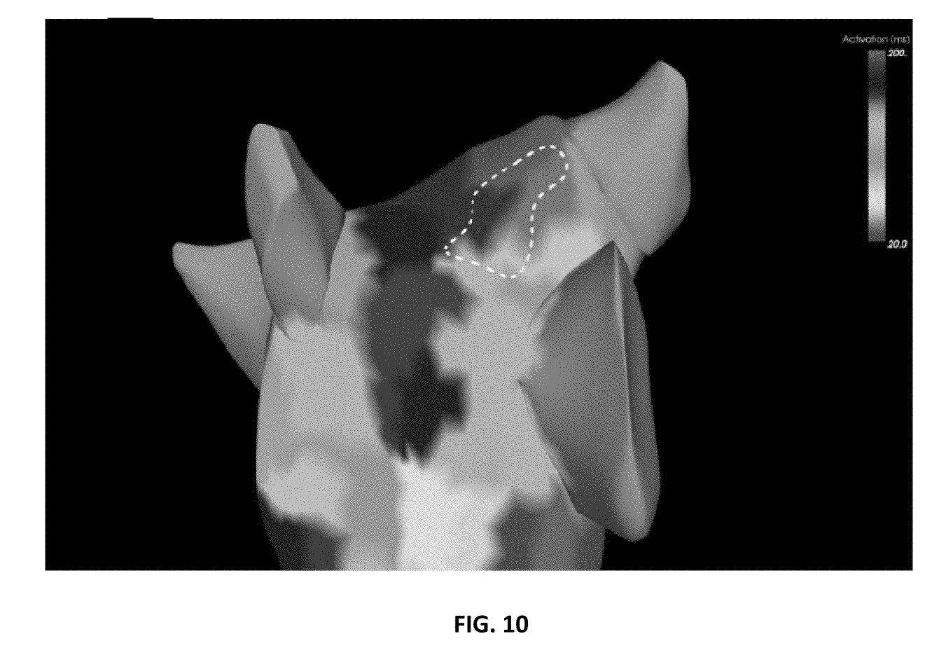

In some instances, all of the electrodes of a catheter are simultaneously in contact with, for example, an atrial heart chamber wall. For example, FIG. 10 illustrates an electroanatomical rotor map projected on 3-dimensional geometry, with all of the electrodes of the catheter in contact with the atrial heart chamber wall. With the basket catheter electrode locations known, the rotor map can be projected onto a 3-dimensional rendering of the heart chamber wall, as shown in FIG. 10. Such rotor mapping projected onto 3-dimensional geometries can reduce and/or limit errors due to, for example, faulty interpretation of a 2-dimensional map. Further, such rotor mapping projected onto 3-dimensional geometries can promote efficient and accurate treatment of certain conditions, such as, for example, atrial fibrillation.

In addition to or instead of receiving diagnostic information delivering therapy to cardiac tissue as described above, in some embodiments, methods, apparatus and/or systems are used to measure rotation, position, angle (e.g., roll angle for deflectable catheters) and/or movement of a catheter. For example, in instances in which a catheter has an adjustable distal end portion, an operator can use the roll angle of the catheter to determine a direction the catheter will bend when an operator initiates a particular action and/or movement of the catheter. Further, various useful information such as force and/or ultrasound imaging can be measured and/or predicted based on such roll angle information, e.g., when such information is formatted and/or incorporated with an electropotential mapping system display.

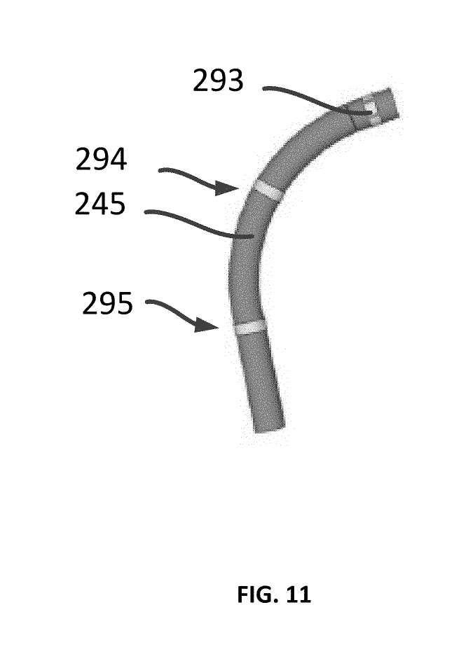

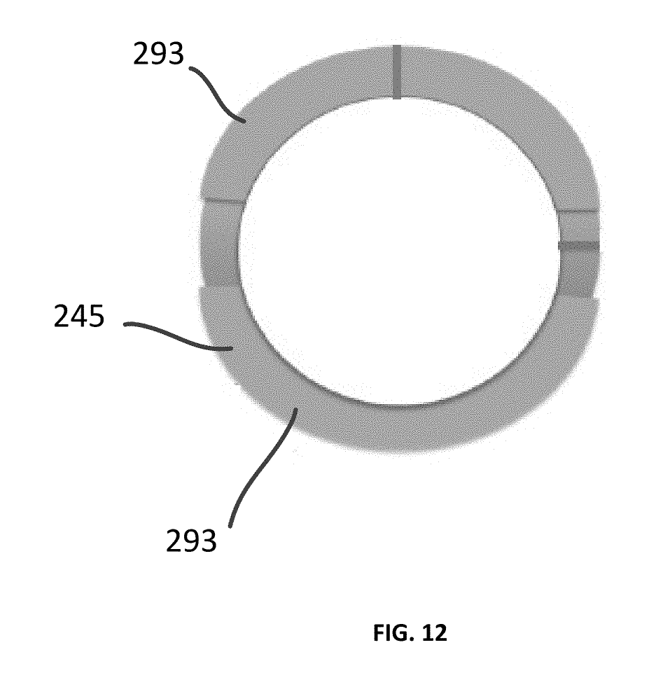

In some embodiments, a roll angle is determined by segmenting a catheter ring electrode into separate radial electrodes. With separate (e.g., physically distinct) radial electrodes, an EP localizer can determine a location of each radial electrode, and determine rotation and/or roll angle of the catheter as it is disposed in and/or moved through the body. FIGS. 11 and 12 illustrate such a catheter having multiple radial electrodes separate from each other disposed about the catheter 245, according to an embodiment. FIG. 11 shows in side view a distal portion of the catheter 245 having separate radial electrodes 293 (i.e., each of the radial electrodes are physically separated from the remaining radial electrodes, and all of the radial electrodes are circumferentially distributed about the catheter). FIG. 12 shows a cross-sectional view of the distal portion of the catheter 245. Although a particular number of separate radial electrodes are illustrated in this embodiment, in alternative embodiments, any suitable number of separate radial electrodes can be used and disposed about a catheter. In addition to the separate radial electrodes (e.g., electrodes 293), a catheter can include any suitable number of additional electrodes (e.g., separate radial electrodes and/or cylindrical electrodes). For example, as shown in FIG. 11, the catheter 245 further includes two cylindrical electrodes 294, 295. Although in this embodiment the catheter 245 includes a single set of radially separate electrodes and two cylindrical electrodes, in alternative embodiments, a catheter can include any suitable number of radially separate electrodes and/or any suitable number of cylindrical electrodes.

With location data generated at and/or provided by the separate radial electrodes 293, the cylindrical electrodes 294, 295, and with knowledge of the characteristics of the catheter 245 (e.g., with using only electropotential localization), a full six degree of freedom location of

As described in previous embodiments, a geometric model of the patient's body, e.g., the patient's heart, can be created and/or rendered at a display device to help an operator suitably manipulate one or more catheters and/or other medical instruments within the body, and to target particular regions therein (e.g., to ablate a target tissue). Some methods of creating geometric models include acquiring and/or defining a point could representation of the body (e.g., the heart chamber) and then manually segmenting the representation into various regions and/or point clouds. The segmented point clouds can be used to produce a representation of the heart chamber, for example, however the manual process used can introduce inaccuracy and subjectivity into the model, thereby leading to a misrepresentation of the patient's actual heart chamber. In some embodiments, to create a better representation of, for example, a patient's heart chamber, systems, apparatus and methods are used to automatically segment using a deformable template, thereby providing for a more accurate geometric model.

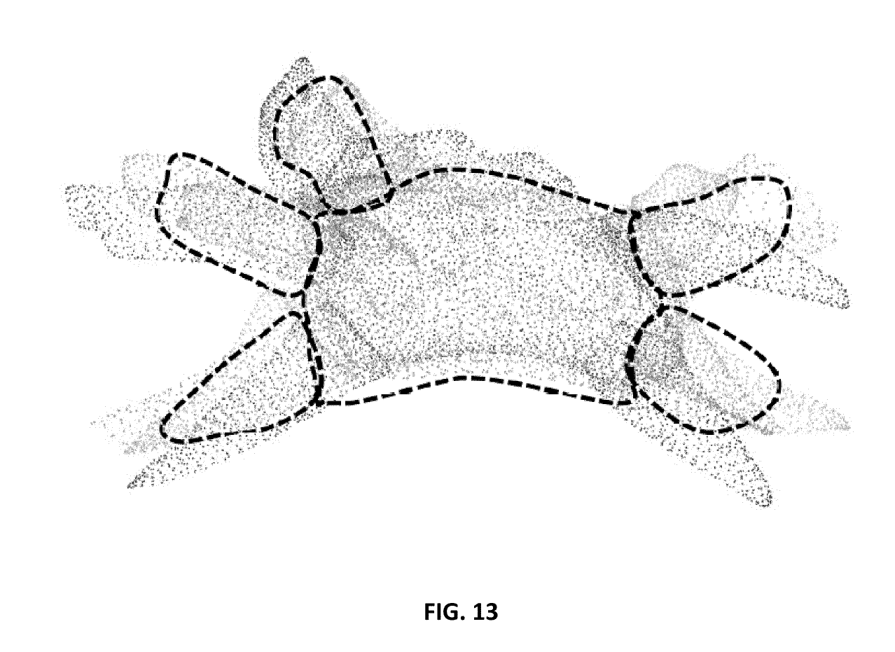

Such a deformable template created based at least in part upon a point cloud data set is illustrated in FIG. 13, according to an embodiment. At least a portion of the data making up the deformable template is provided by or derived from actual patient data acquired during a previous electropotential procedure. The anatomic model segments of the template are configured to interact with each other and the point cloud acquired during the electropotential procedure. In this manner, the template is deformed to fit the point cloud by balancing a force interaction equation to equilibrium through a recursive process, as described in more detail herein.

The template segments interact with each other and the point cloud it surrounds through an application of force to each other. A shape is determined by all the forces being in equilibrium, as defined by the following force equation:

Σ\vec{F}=\vec{F}_{c}+\vec{F}_{pc}+\vec{F}ₚₘ=0

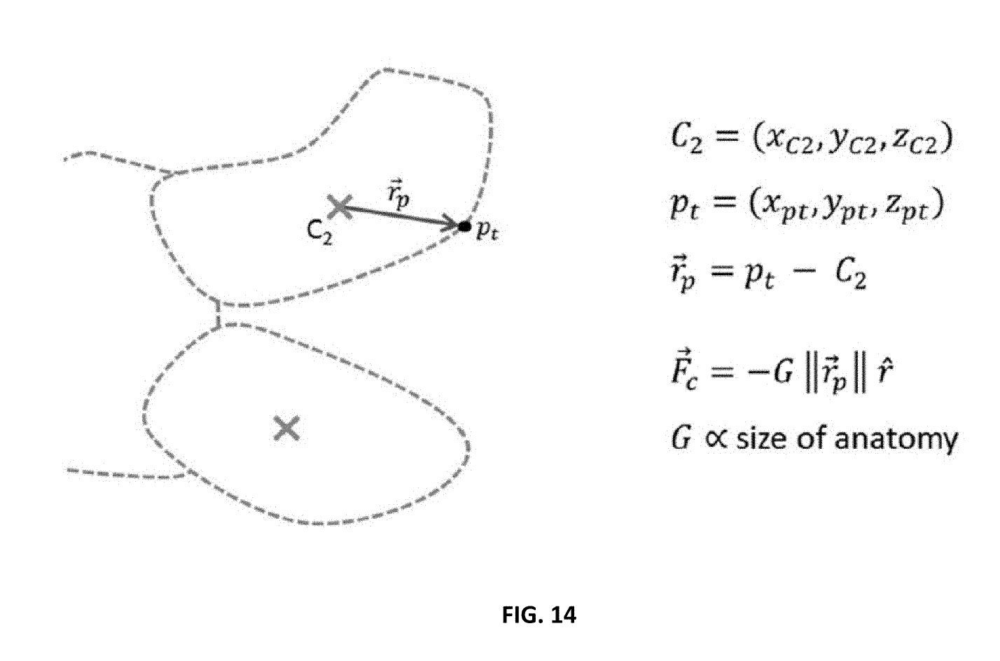

Fc represents an attractive force of a point on a surface of the sub-model segment to the centroid of the segment, pt. The force pulling on the point on the surface toward the centroid increases as the distance between the point and the centroid increases. FIG. 14 illustrates (on the left) an attractive force exerted by the centroid of the model template, Fc, and (on the right) shows equations for determining the attractive force of the centroid of the template.

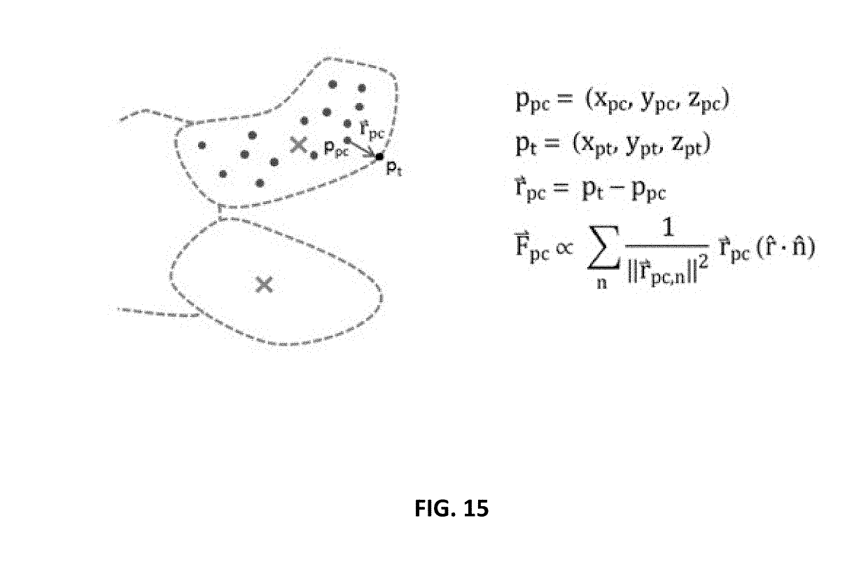

FIG. 15 illustrates (on the left) a repulsive force of the point cloud to the model template surface, and (on the right) shows equations for determining the repulsive force. Fpc represents the repulsive force of the point cloud, ppc, to the point on the surface of the sub-model segment, as shown in FIG. 15.

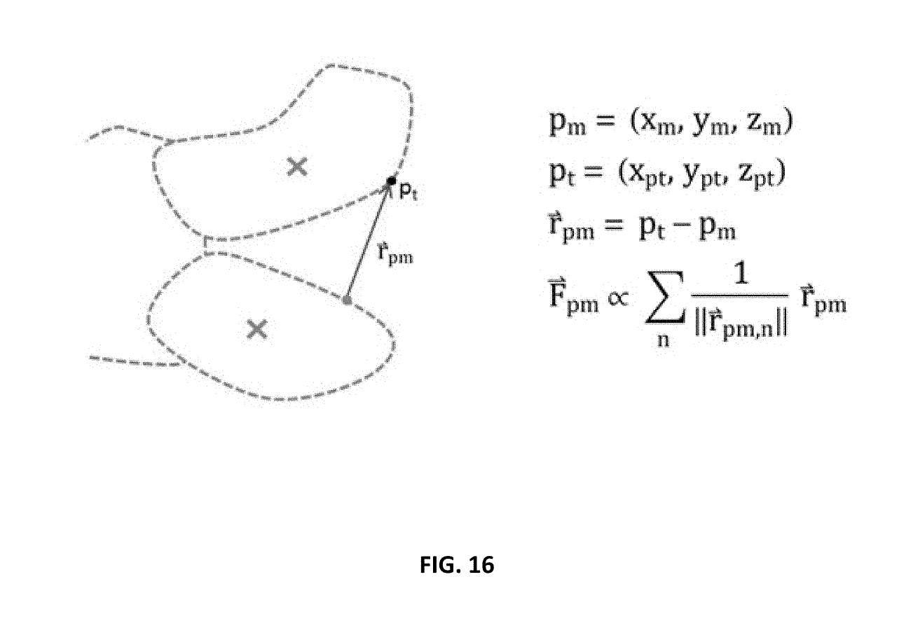

FIG. 16 illustrates (on the left) a repulsive force of the sub-model segments pushing against each other, and shows (on the right) equations for determining the repulsive force of the sub-model segments.

The surface of the template is deformed by calculating the forces acting on the individual surface points. The new position of each surface point is determined based at least in part on the following linear kinematic equation of motion:

$$\vec{a} = {μ}_{m} \frac{{\vec{F}}_{c} + {\vec{F}}_{pc} + {\vec{F}}_{pm}}{m}$$

$$\vec{r} ( t ) = {\vec{r}}_{o} + {\vec{v}}_{o} t + \frac{1}{2} \vec{a} {t}^{2}$$

The acceleration equation includes a malleability constant. The malleability constant is a unit-less value proportional to a temperature of the template to room temperature. The malleability constant is included in the acceleration equation to allow the template to be more malleable at the beginning of the process, and then slowly becoming more rigid until a final shape of the template is determined. In this manner, the malleability constant limits the degree to which the template can deform and/or reduce the time to stabilize the geometry of the template. This process can stop, for example, when the template surface points cease to move beyond a threshold value (e.g., case to move significantly) between iterations.

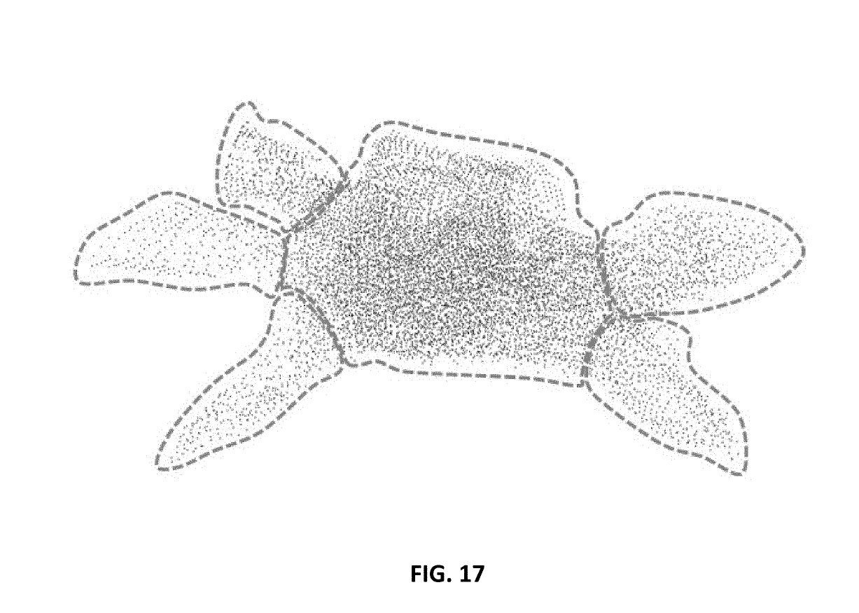

With the template in its final shape or configuration, the portion of the point cloud that is inside the surface of the each template segment is considered to be a part of that segment, as illustrated in FIG. 17. In this manner, the point cloud is segmented into the correct chamber segments (e.g., corresponding to the patient's heart chamber), and can be used to create a graphical representation of the patient's anatomical geometry used in the procedure. As such, the point cloud acquired during the procedure can be segmented automatically and efficiently without undesirable subjectivity of a medical technician or their imperfect ability to properly discern different anatomical region of the patient's heart.

In some embodiments, cardiac information, collected for by a system such as, for example, the system shown and described with respect to FIGS. 1-7, may be used to identify the optimal site to place a pacemaker or defibrillator lead or an entirely miniaturized pacemaker or defibrillator to ensure optimal current flow from the stimulation source through diseased or scarred tissue to stimulate the heart in a manner that resonates with sinus rhythm as opposed to introducing current flow patterns that may cause another unwanted arrhythmia. During the surgery, additional measurements may be taken from a tracked internal instrument (e.g., catheter 45, catheter 145, etc.) to further understand patient characteristics internally, such as voltage transitions indicating scar tissue, and that information can be merged with the original model of the heart and the associated body surface electrogram information. This combined information can be used to tune the output settings of a pacemaker or defibrillator based on external and internal information of the patient. Similar methods of treatment delivery can be constructed for biological drug delivery such as nano-particles, stem-cells, gene therapy.

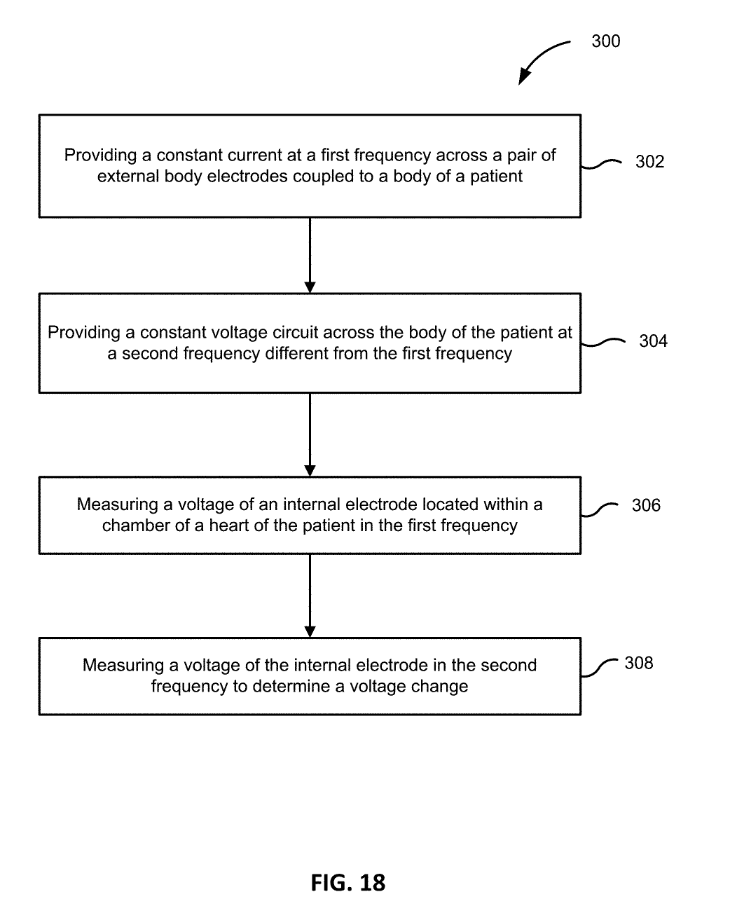

FIG. 18 shows a schematic flow diagram of a method 300 for electro-anatomical mapping of a catheter. The method 300 includes providing a constant current at a first frequency across a pair of external body electrodes coupled to a body of a patient, at 302, and providing a constant voltage circuit across the body of the patient at a second frequency different from the first frequency, at 304. The method 300 further includes measuring a voltage of an internal electrode located within a chamber of a heart of the patient in the first frequency, at 306. The method 300 further includes measuring a voltage of the internal electrode in the second frequency to determine a voltage change.

It is intended that the systems and methods described herein can be performed by software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code. Each of the devices described herein can include one or more processors as described above.

Some embodiments described herein relate to devices with a non-transitory computer-readable medium (also can be referred to as a non-transitory processor-readable medium or memory) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

Although various embodiments have been described as having particular features and/or combinations of components, other embodiments are possible having a combination of any features and/or components from any of embodiments as discussed above.

1. A system, comprising:
a pair of external body electrodes;
a first control unit configured to (1) provide constant current at a first frequency across the pair of external body electrodes coupled to a body of a patient, and (2) provide a constant voltage circuit across the body of the patient at a second frequency different from the first frequency; and
a second control unit configured to (1) measure a voltage of an internal electrode located within a chamber of a heart of the patient in the first frequency, and (2) measure a voltage of the internal electrode in the second frequency to determine a voltage change.

2. The system of claim 1, wherein the voltage change is based on contact between the internal electrode and body tissue of the heart.

3. The system of claim 2, wherein the voltage change corresponds to a surface area of the internal electrode being imbedded in a wall of the heart.

4. The system of claim 1, wherein at least one of the first control unit or the second control unit is configured to define a correlation table to map a force required to imbed the internal electrode into the wall of the heart to generate the voltage change.

5. The system of claim 1, wherein at least one of the first control unit or the second control unit is configured to define a correlation table to map ablation lesion size when ablation is performed with the corresponding voltage change of the internal electrode.

6. A system, comprising:
a trackable medical instrument operably coupled to a control unit,
the control unit configured to (1) receive positional data from the trackable medical instrument when the trackable medical instrument is disposed within a patient's heart chamber, (2) generate a cloud of points in at least three dimensions based on locations visited by the trackable medical instrument within the heart chamber, and (3) modify a template three-dimensional surface model of a generic heart chamber based on interactive forces between the cloud of points and the template three-dimensional surface model.

7. The system of claim 6, wherein the control unit is configured to translate, rotate, scale, and stretch the template three-dimensional surface model of the generic heart chamber based on the interactive forces between the cloud of points and the template three-dimensional surface model.

8. A method, comprising:
collecting a plurality of location points within a patient's heart anatomy;
calculating an attractive force of points in the plurality of location points to a centroid of a template;
calculating a repulsive force of the plurality of location points to the template, wherein the template includes a plurality of template regions;
recursively balancing the attractive force and the repulsive force to equilibrium;
overlaying a modified template over the plurality of location points; and
segmenting a plurality of point clouds based on the modified template.

9. The method of claim 8, further comprising:
capturing the plurality of location points with an ultrasound imaging device having an integrated electromagnetic sensor.

10. An apparatus, comprising:
an elongated cylindrical catheter with an array of electrodes aligned radially around the outer circumference of the catheter, at least two electrodes from the array of electrodes being partially wrapped around opposite ends of the circumference of the catheter, each electrode from the array of electrodes being independently connected to a navigation system; and
a control unit configured to (1) receive position data of each electrode from the array of electrodes, (2) define a vector orthogonal to a center axis of the catheter, and (3) calculate a roll of the catheter orientation.

11. The apparatus of claim 10, wherein the catheter includes at least electrode that is continuously circumferentially disposed about the catheter.

12. The apparatus of claim 10, wherein the control unit is configured to determine a location in three-dimensions of each electrode from the array of electrodes, the control unit configured to determine a six-degree of freedom location of the catheter based on the location in three-dimensions of each electrode from the array of electrodes.

13. The apparatus of claim 10, wherein the control unit is configured to determine a rotation of the catheter, the control unit configured to send to a display device a signal representing the rotation of the catheter such that a graphical representation of the rotation of the catheter is displayed on the display device.

14. The apparatus of claim 13, wherein the control unit is configured to determine a directional force based on data generated by the array of electrodes, the control unit is configured to send to the display device a signal representing the directional force such that a graphical representation of the directional force is displayed on the display device.

15. The apparatus of claim 14, wherein the control unit is configured to send to the display device a signal representing ultrasound image information such that a graphical representation of the ultrasound image information is displayed on the display device.
